(19) United States
(12) Patent Application Publication
(10) Pub. No.: US 2010/0022907 A1
(43) Pub. Date: Jan. 28, 2010

(54) Methods based on fluctuations in cortical synchronization

(75) Inventors: Jose Luis Perez-Velazquez, Toronto (CA); Vera Nenadovic, Toronto (CA); Luis Garcia Dominguez, Toronto (CA)

Correspondence Address: VALENTINE A COTTRILL;SUSAN TANDAN, 50 QUEEN STREET NORTH, STE. 1020, P.O. BOX 2248, KITCHENER, ON, N2H6M2, CA

(73) Assignee: The Hospital for Sick Children, Toronto (CA)

(21) Appl. No.: 12/453,528

(22) Filed: May 14, 2009

Related U.S. Application Data
(60) Provisional application No. 61/071,721, filed on May 14, 2008.

(51) Int. Cl. A61B 5/0476

(52) U.S. Cl. 600/544

(57) ABSTRACT

A method of identifying a site of injury in a pediatric patient with brain injury is provided which comprises obtaining an electroencephalogram signal on the patient. The identification of phase synchrony within the signal is indicative of the site of brain injury. Electroencephalogram signals may also be used to determine prognosis of a pediatric patient in a coma in which an increase in the temporal variability of phase synchronized EEG signals over time is indicative of an improvement in the patient.

### FIELD OF THE INVENTION

The present invention relates generally to methods of diagnosis and prognosis with respect to conditions of the pediatric brain, including brain injury and coma, based on fluctuations in cortical synchronization.

### BACKGROUND OF THE INVENTION

Traumatic brain injury remains the leading cause of death and acquired disability in the paediatric population worldwide. The sequelae of traumatic brain injury have implications for the child's family functioning, educational and social development. Accurately predicting outcome would enable clinicians to anticipate consequences, thereby focusing treatment and rehabilitation and potentially improving long-term outcome. The complexity of the brain likely precludes a simple model or single diagnostic tool for accurate prediction. Various prediction models have been examined in children utilizing combinations of clinical parameters, electrophysiology and neuroimaging, but to date no practical model exists.

Neurophysiological activity is altered following traumatic brain injury resulting, in the initial phases of post-injury, in neuronal hyperexcitability. The electroencephalogram recordings reveal a generalized slowing of brain frequencies to the delta and theta ranges. These bandwidths predominate masking the higher frequency and lower amplitude waves that may be present and may be necessary for recovery following traumatic brain injury. There are time dependent alterations in synaptic function following cortical injury and structural damage with subsequent cell reorganization, which may be reflected in the electrophysiology of the brain.

Electrophysiological analysis of patients with cerebral trauma and concussion was first reported in the 1970's. The synchrony of electroencephalogram signals, or coherence, was evaluated in a few studies and thought to reflect neuroanatomical inhomogeneities corresponding to features of neocortical cytoarchitecture and axonal fibre systems. It was proposed that the analysis of the coherence of post traumatic electroencephalogram waves may detect and quantify diffuse axonal injury. Low frequency brain rhythms have been observed after head trauma and a study reported that 65% of patients with mild traumatic brain injury had dipolar clusters of low frequency activity recorded with magnetoencephaolography.

Synchrony analysis, specifically phase synchronization based on the analytic signal approach, has evolved as the power and utility of computers has improved, and is now more sophisticated than previous analysis of coherence. It is emerging as a method of measuring brain function in patients with other pathologies, such as epilepsy and schizophrenia. Normal brain function is believed to result from fluctuating patterns of synchronization and desynchronization between neuronal networks. These fluctuations are a reflection of the information processing occurring in the brain networks.

In view of the foregoing, it would be desirable to develop an understanding of brain function during injury, and utilize this knowledge for diagnostic or prognostic purposes.

### SUMMARY OF THE INVENTION

It has now been found that brain synchrony patterns are altered following pediatric brain injury, and correlate with site of injury. It has also been found that brain synchrony patterns are altered in pediatric coma patients, and are indicative of patient prognosis.

Thus, in one aspect of the invention, a method of identifying a site of injury in a pediatric patient with brain injury is provided comprising the steps of:
(a) obtaining an electroencephalogram signal on the patient; and
(b) analyzing phase synchronization from the signal obtained, wherein synchrony in the sample is indicative of a site of brain injury.

In another aspect of the invention, a method of prognosis in a pediatric patient in a coma is provided comprising the steps of:
(a) obtaining a first electroencephalogram signal on the patient subsequent to the onset of coma;
(b) obtaining a second electroencephalogram signal on the patient at a time subsequent to obtaining the first signal;
(c) analysing phase synchronization patterns in the first and second signals, or in a portion of the first and second signals; and
(d) calculating the temporal variability of the synchronized patterns, wherein increased variability in the signal over time is indicative of an improvement in the patient.

These and other aspects of the invention will become apparent by reference to the detailed description that follows and to the following figures.

### BRIEF DESCRIPTION OF THE FIGURES

FIG. 1 depicts the transformation of a raw EEG signal in referential montage (A) to a two dimensional synchrony diagram (B);

FIG. 2 illustrates the power spectral analysis comparing frequencies over the 10 second epoch between a control subject with eyes closed (left) and patient with eyes closed (right);

FIG. 3 is a comparison of EEG synchrony of patient (top) and control subject (bottom) at 3, 6, 15 and 25 Hz;

FIG. 4 illustrates the placement of electrodes on the head of a patient;

FIG. 5 illustrates the raw EEG traces and synchrony graphs of a patient with good outcome (A), bad outcome (B) and a control subject (C) are presented; and

FIG. 6 illustrates the distribution of temporal variability of EEG synchrony of controls (solid black squares at zero on the x axis) compared to patients who are grouped based on their outcome (PCPC) scores.

### DETAILED DESCRIPTION OF THE INVENTION

A method of identifying a site of injury in a pediatric patient with brain injury is provided. The method comprises obtaining an electroencephalogram (EEG) signal on the patient over a time period; and calculating the phase synchronization of the signal obtained. Synchrony within the sample is indicative of a site of brain injury.

Synchrony is defined as the adjustment of rhythms of oscillations that are weakly coupled. The synchronization index, R, quantifies this adjustment as the degree of phase locking between two time series. Desynchrony occurs when the synchronization index (R) is a null value (R=0) as there is no synchrony between two time series.

Phase synchronization is defined as the process by which two or more cyclic signals, in this case EEG channel signals, tend to oscillate with a repeating sequence of relative phase angles. Phase synchronization is quantified by the calculated synchronization index (R value).

The term “pediatric patient” refers to a patient of no more than about 17 years old.

The present method includes obtaining an electroencephalogram signal on a pediatric patient using procedures and equipment well-established in the art. Although a continuous electroencephalogram is preferable for evaluating synchrony patterns as will be described, EEGs obtained for a time period within a range of about 15-45 minutes, preferably about 25-35 minutes, may be used. The EEG signals are recorded at an acquisition rate of at least about 150 Hz.

Phase synchronization of EEG signals is determined using signal transformations. Initially, a selected region of each EEG signal is processed to provide a reference-free signal, for example using a Laplacian transform. Instantaneous phases of each signal are then extracted using, for example, the Hilbert transform. Prior to this phase extraction step, the signals may be passed through a bandpass finite impulse response filter, for example, a bandpass having a width of about 4 Hz around a central frequency. Phase synchronization is then evaluated as described in the specific examples herein at a discrete number of central frequencies within the range of about 1 to 30 Hz. As a result of potential interference from medication, EEG synchrony is preferably evaluated at a frequency in the range of about 1 to 15 Hz, and in particular, in the delta frequency range of 1-5 Hz, to minimize such interference.

Synchrony within the EEG of a pediatric patient has been found to be indicative of the site of brain injury in comparison to control EEGs. As one of skill in the art will appreciate, a degree of synchrony exists in control EEGs as well; however, synchrony that is at least 2 standard deviations above the mean synchrony calculated based on control data is associated with the diagnosis of site of pediatric brain injury. In this regard, the statistical method of calculating surrogate data may be used to determine the significance of synchrony in an EEG signal. Surrogate data is calculated by shuffling the time series and comparing the shuffled time series to the original. If the shuffled time series does not exhibit significant synchrony, e.g. of at least about 2 standard deviations above the mean, then the observed synchrony is significant and indicative of site of injury.

In another aspect of the invention, a method of prognosis for a pediatric patient in a coma is provided. The coma-induced state may be as a result of any one of brain injury, for example due to head trauma, metabolic abnormalities, liver or kidney failure, hypoxia, imbalance of electrolytes, central nervous system infections, brain hemorrhage, seizure disorders, cardiac arrest, stroke, extreme elevation of blood pressure, and exposure to toxins, including poisons, alcohol and other drugs (e.g., barbiturates, opiate narcotics, sedatives, amphetamines, cocaine, aspirin).

This prognostic method comprises comparing a first phase synchronized electroencephalogram signal obtained from the patient subsequent to the onset of coma with a second phase synchronized electroencephalogram signal obtained from the patient at a time subsequent to the first signal; and calculating the temporal variability of the phase synchronized signals. The EEG signals are obtained as described above for a period of time in the range of about 15 to 45 minutes and at an acquisition rate of at least about 150 Hz. Phase synchronization is also conducted as described above, preferably at a frequency in the range of 1-30 Hz.

The first EEG signal is obtained as soon as possible following onset of coma, and preferably within a range of about 24-48 hours following coma onset. The second EEG signal is obtained at a time subsequent to the first EEG signal, and preferably within a range of about 48-72 hours following onset of coma. As one of skill in the art will appreciate, it may not be possible to obtain EEG readings within these preferred ranges. EEG signals obtained outside of the preferred ranges are also useful, for example, the first EEG signal may be obtained at any time subsequent to onset of coma, up to about 4-5 days or more, and the second EEG signal may be obtained up to about 8-10 days following injury. Preferably, the second EEG signal is obtained about 18-48 hours following the time of the first EEG signal, and more preferably within about 18-36 hours following the time of the first signal, but may be obtained up to about 7 days or longer following the first EEG signal.

The present prognostic method may advantageously be used to monitor patient progress at multiple times following onset of coma in a pediatric patient, or even continuously, by obtaining multiple EEGs and calculating temporal variability as new EEG signals are obtained, by comparing the most recently obtained EEG signal with a previous EEG signal.

Temporal variability in phase synchronized EEG signals is the variance of a temporal sequence of synchrony values. The result of the evaluation of the temporal variability in the synchrony patterns may be an average of the variance over a certain time period, which will vary from case to case, or a time series of the variance. In more technical terms, temporal variability in phase synchronized EEG signals is evaluated as the mean value of the absolute derivative of a series of synchronization values. Alternatively, the temporal variability in phase synchronized EEG signals may be evaluated as the mean value of the square of the derivative of such series. Spatial complexity is a related measure that quantifies how tightly the phase synchronization values cluster around a single mean value. If the brain demonstrates highly coordinated activity (phase synchrony) for a time epoch, then the cluster will be tight, indicating that for the particular time series there is an almost constant phase difference and therefore little variability. In this instance, spatial complexity would be deemed to be lower, and the distribution of EEG phase synchrony is highly predictable. In contrast, a high degree of variability with rapid fluctuations between phase synchrony and desynchrony represents increased spatial complexity which is another measure of variability of the EEG phase synchronization.

An increase in the temporal variability of the phase synchronized EEG signal over time is indicative of an improvement in the patient, e.g. emergence from coma, and thus, a good prognosis. In this regard, the amount of increase in the temporal variability that is indicative of patient recovery varies with the degree of recovery of the patient, and thus, may be an increase in the range of about 10%-100%. For example, a change in level of coma from completely comatose (Glascow coma score (GCS)=3) to completely awake (GCS=15) may be indicated by an increase in temporal variability of 100%, while smaller changes in the level of coma, such as from a GCS 8 to GCS 10, may be indicated by a much smaller increase in temporal variability.

The use of EEG synchrony to determine site of pediatric brain injury, and the use of temporal variability of EEG synchrony for prognosis in cases of pediatric coma, provide valuable non-invasive diagnostic and prognostic tools. The ability to non-invasively determine site of brain injury provides a means to more effectively treat the injury, if possible. In addition, the measure of temporal variability of EEG synchrony calculations from electroencephalography provides a non-invasive means to more frequently analyze patient progress, or a means to continuously monitor patient progress in terms of emergence from coma.

Embodiments of the invention are described in the following specific example which is not to be construed as limiting.

### Example 1

### Materials and Methods

### Patient Population and Clinical Data

Patients were included if they were admitted to the Critical Care Unit at the Hospital For Sick Children, Toronto, Canada with traumatic brain injury and parental consent was obtained. Exclusion criteria were: suspected brain death, penetrating trauma as a cause of the head wound and parental refusal of consent. Control subjects were also recruited who had no history of seizures, no current neurological conditions, no history of head injury and no psychiatric conditions. The study was approved by the Hospital for Sick Children Research Ethics Board.

Each patient had baseline data of age, gender, mechanism of injury, pediatric risk of mortality score recorded and Glasgow coma scale was recorded on admission and at the time of each electroencephalogram. A computerized tomography scan was performed on admission and repeated as deemed necessary by the attending intensive care physician or neurosurgeon and were evaluated by a neuro-radiologist.

### Electroencephalogram Recordings

All patients had scalp electroencephalograms done for 30 minutes within the first 60 hours of their admission and repeated before day 5 of their admission. Control subjects also had a thirty minute scalp electroencephalogram done in the outpatient clinic. The electroencephalograms were acquired using the 32 channel XLTek EEG32 portable electroencephalography system (XLTek, Oakville, Ontario) and standard 10-20 montage with PZ prime as the reference electrode. Bandpass width of 1-70 Hz and a 60 Hz notch filter was used with a sampling frequency of 499 Hz. Each electroencephalogram was evaluated by a certified electroencephalographer and reported to the attending intensive care physician.

### Outcome Measures

Pre-injury function was assessed in the first week post-injury by parental interview, and post-injury function was assessed at 12 months, using the paediatric cerebral performance category (PCPC) score, a validated paediatric critical care outcome score as described in Fiser (J Pediatr.1992.121(1)), the contents of which are incorporated herein by reference, particularly pages 68-74. It is a 6 point score where 1=normal function, 2=mild disability, 3=moderate disability, 4=severe disability, 5=coma or persistent vegetative state, and 6=death.

### Phase Synchrony Analysis and Calculation of the Temporal Variability of Synchronization

A ten second electroencephalogram epoch was extracted from every 6 minute electroencephalogram segment for a total of 4 epochs for each patient and each control subject. The patients' epochs were free of eye movement, muscle artefact or extraneous artefact (eg. ventilator, intravenous drip or electrocardiogram artefact). In the control electroencephalograms, the segments represented a quiet state with eyes closed. Sleep and drowsy episodes were excluded from the synchrony analysis in both patients and controls, though the presence of sleep features was noted. The selected segments were exported in the referential montage as text files. Power spectral analysis was initially done for each patient and the control subject electroencephalograms.

Initially four electroencephalogram frequencies were evaluated: delta (3 Hz±2 Hz), theta (6Hz±2 Hz) and two beta frequencies (15Hz±2 Hz and 25 Hz±2 Hz). Potential confounding effects of medications used in the treatment of TBI patients in the critical care unit made the higher beta bandwidth more problematic. Preliminary analysis of power spectra and synchrony was performed at all four frequencies. Detailed analysis of EEG synchrony and temporal variability of EEG synchrony was confined to the delta and lower beta frequencies. All four frequencies were evaluated in the control subjects as they would have no confounding effects of medication.

Medications that affect electroencephalography recordings are commonly used in the paediatric critical care unit. Phenytoin is frequently used post TBI as seizure prophylaxis. Benzodiazepines such as lorazepam and midazolam are used to treat seizures and as sedatives. An increase in the higher beta frequencies (18 to 25 Hz) is seen in electroencephalography recordings with use of benzodiazepines. The background alpha frequency (7 to 12 Hz) is affected by phenytoin and also by patient age, with young children having lower alpha frequencies than older children and adults. The present patient group was heterogeneous with respect to use of sedatives, seizure prophylaxis and with respect to age. To avoid the potential confounding factor of medication use and age, for the purposes of electroencephalogram synchrony correlation with CT scan findings and for evaluation of temporal variability of EEG synchrony, two frequencies were studied: lower beta (15±2Hz) and delta (3Hz±2 Hz).

The synchrony of the electroencephalography signal between each electrode and the other 18 electrodes was calculated using, first, a Laplacian transform, and then the instantaneous phases were extracted using the Hilbert transform. Electroencephalograms were first processed using a Laplacian transform to avoid the potential effects of the reference electrode on synchronization by mathematically approximating a reference-free signal as described in Guevara et al. (Neuroinformatics. 2005. 3(4)), the contents of which are incorporated herein by reference, particularly pages 301-314). Next, all signals were band-passed with an order 100 Constrained Least Square Finite Impulse Response filter (FIRCLS) (f±2Hz) prior to the extraction of the instantaneous phases using the Hilbert transform. The Hilbert Transform is particularly useful for analyzing the electroencephalogram whose waveforms are nonstationary and noisy and have multiple frequencies that change over time by extracting the instantaneous phase of the signal (Burns, 2004). Phase synchronization is then calculated as the degree of phase locking between two channels using the circular variance of the phase difference distribution

$$R = \vert \frac{1}{N} {\sum }_{j = 1}^{N} {e}^{iΔα ( jΔ t )} \vert$$

where |.| denotes absolute value and N is the number of data points that are being considered (Mormann et al., 2000). Phase locking being the condition where the phase difference of the two oscillators m, n remains constant or nearly constant during a given period of time: Δα(t)=αₙ(t)−αₘ(t). αₙ(t) denotes the instantaneous phase of signal n. Although the general condition should include any multiples of the individual phases, only the 1:1 relation as stated above was assessed for simplicity in this study. A time window of 1 second was used. To avoid the spurious synchrony based on two signals having similar individual (univariate) properties, surrogate R values were calculated after shifting one time series in relation to the other. The shifting was random and taken from a flat distribution. In this way, individual time series properties were retained (i.e. power spectrum) while synchronization was disrupted. The phase synchronization between the data sets is significant if the R values for the unshifted phase angles are greater than two standard deviations from the mean calculated from 100 time shift surrogates. The departure from the surrogate mean of R in standard deviations is denoted by S. The S values for each individual channel were then compared to each of the remaining channels to establish which electroencephalography channels were synchronized over the time series. This provided information on which corresponding brain regions underlying the EEG electrodes would be synchronized.

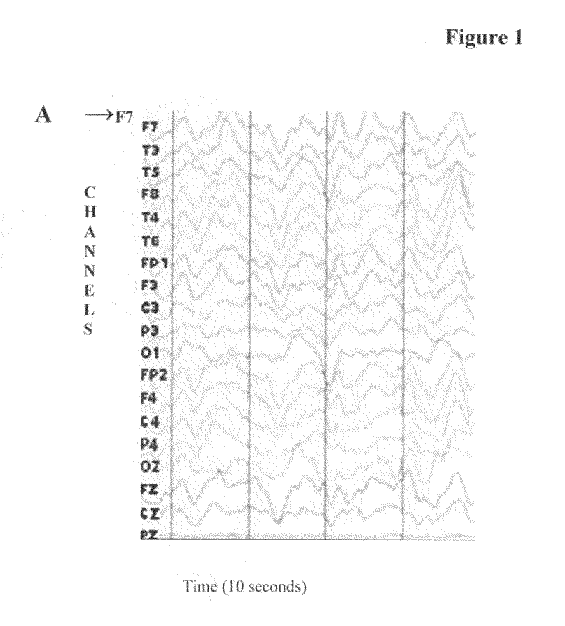

The temporal variability of the synchronization was calculated as the mean value of the absolute temporal derivative of S. Temporal variability was calculated for each of the two electroencephalograms for each patient and for the one electroencephalogram of the control subjects. FIG. 1 depicts the transformation from raw electroencephalography signal (A) to a synchrony graph (B). The raw EEG signal shows the 19 scalp channels over the 10 second epoch. The synchrony graph transformation depicts the S values of each channel compared with the remaining other eighteen channels, plotted over the 10 second epoch. The bar interprets the colour scheme where dark red is maximal synchrony and dark blue is maximal desynchrony. The two dimensional head plot (C) depicts the same synchrony as in (B), but with the EEG channels plotted on the scalp as black dots. The white dot (arrow highlight) represents the first channel as seen in A and B, which is F7.

### Statistical Analysis

Repeated measures analysis of variance evaluated the within subject variance for the four 10-second epochs of each patient and control subject electroencephalogram. The S values of individual channels of each patient electroencephalogram were compared with those of control subjects, using a two-tailed Student t-test. A two-tailed Student t test was used to compare the temporal variability of the synchrony of each patient's second electroencephalogram with that of his/her first electroencephalogram. Analysis of variance was also used to evaluate the electroencephalogram synchronization and temporal variability of synchronization among control subjects. Linear regression was used to model Pediatric Cerebral Performance Category (PCPC) score as a function of each of the clinical parameters: initial Glasgow coma scale (GCS); age and Pediatric risk of mortality score. The Pediatric Cerebral Performance Category score was also modelled as a function of the temporal variability calculated for each 10-second electroencephalogram epoch (four per patient) using repeated measures analysis of variance.

### Results

Patient data are summarized in Table 1. No seizures were clinically documented nor found on electroencephalogram.

TABLE 1

| | | GCS at | | |
|---|---|---|---|---|
| | | the | | |
| Gender | Age | scene | Mechanism of Injury | CT Scan Findings |
| F | 12 Y | 3 | Motor Vehicle Collision | Subarachnoid hemorrhage |
| M | 12 Y | 11 | Assault | Subdural hemorrhage |
| M | 10 Y | 8 | Motor Vehicle Collision | Subarachnoid hemorrhage, |
| | | | | Diffuse axonal injury |
| F | 6 Y | 14 | Motor Vehicle Collision | Intraventricular hemorrhage |
| F | 5 Y | 15 | Fall | Hematoma |
| M | 2 Y | 11 | Motor Vehicle Collision | Normal |
| F | 16 Y | 3 | Motor Vehicle Collision | Subarachnoid hemorrhage, |
| | | | | Diffuse axonal injury, |
| | | | | Intraventricular hemorrhage |
| M | 6 Y | 10 | Fall | Subdural hematoma |
| M | 9 M | 9 | Fall | Subdural hematoma |
| F | 6 Y | 15 | Motor Vehicle Collision | Hematoma |
| M | 10 Y | 10 | Motor Vehicle Collision | Subarachnoid hemorrhage |
| M | 3 Y | 8 | Motor Vehicle Collision | Normal |
| M | 14 Y | 7 | Motor Vehicle Collision | Subarachnoid hemorrhage, |
| | | | | Diffuse axonal injury |
| M | 4 Y | 3 | Motor Vehicle Collision | Cerebral Infarct |

GCS—Glasgow Coma Scale score

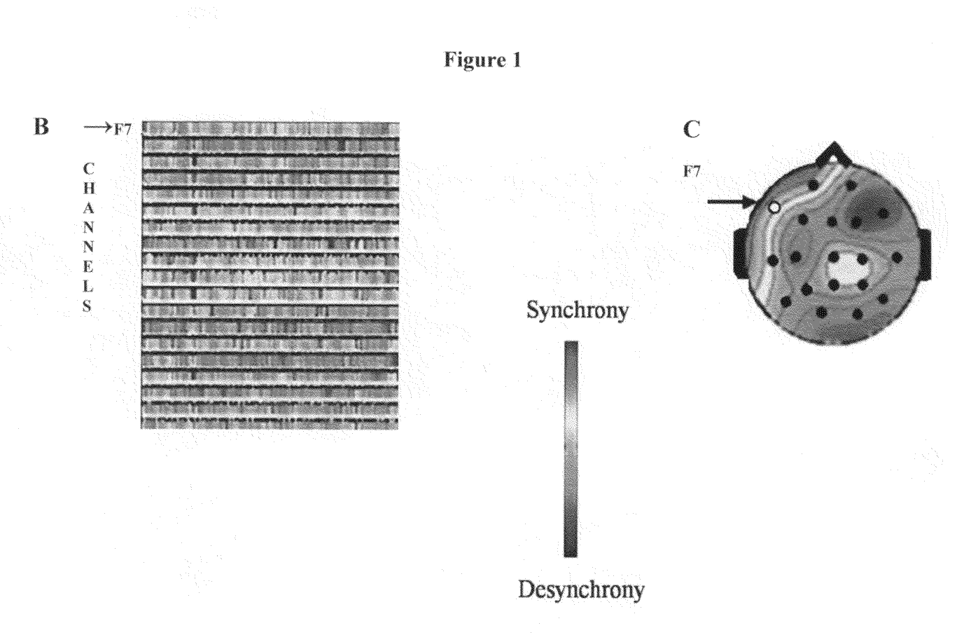

The first electroencephalogram was done at 55.9 hours ±28.7 hours (range: 22.25 to 110 hours) and the second at 115.6±46.7 hours (range: 46 to 242.5 hours) following the injury. The electroencephalograms of the patients with head trauma had a predominance of slower frequencies in the delta (1 to 3 Hz) and theta (4 to 6 Hz) range whereas the control subject electroencephalograms had a predominance of higher frequencies. This was confirmed by power spectral analysis (FIG. 2). The control subject's EEG demonstrates overall lower amplitude with activity in all bandwidths up to 75 Hz (150). The patient's EEG demonstrates higher amplitude in the lower frequency bands, up to 25 Hz (50). There is minimal activity in the higher frequencies and breakthrough of 60 Hz (120) despite the notch filter.

There were no significant differences in synchrony between the four 10-second epochs within the electroencephalograms of patients with traumatic brain injury. The preliminary evaluation of electroencephalogram synchronization was then performed in both patients with trauma and in control subjects at the four bandwidths: central (delta), theta and two beta frequencies. For control subjects, no significant differences in the synchrony between the four 10-second epochs within subjects' electroencephalograms were found. Evaluation of control subjects' differences resulted in a logarithmic distribution with most subjects within one standard deviation of the mean. Only the youngest subject (11 month old boy) temporal variability was >four standard deviations of the mean. This child's EEG was then used for comparison with the youngest patient in the group (9.5 months old).

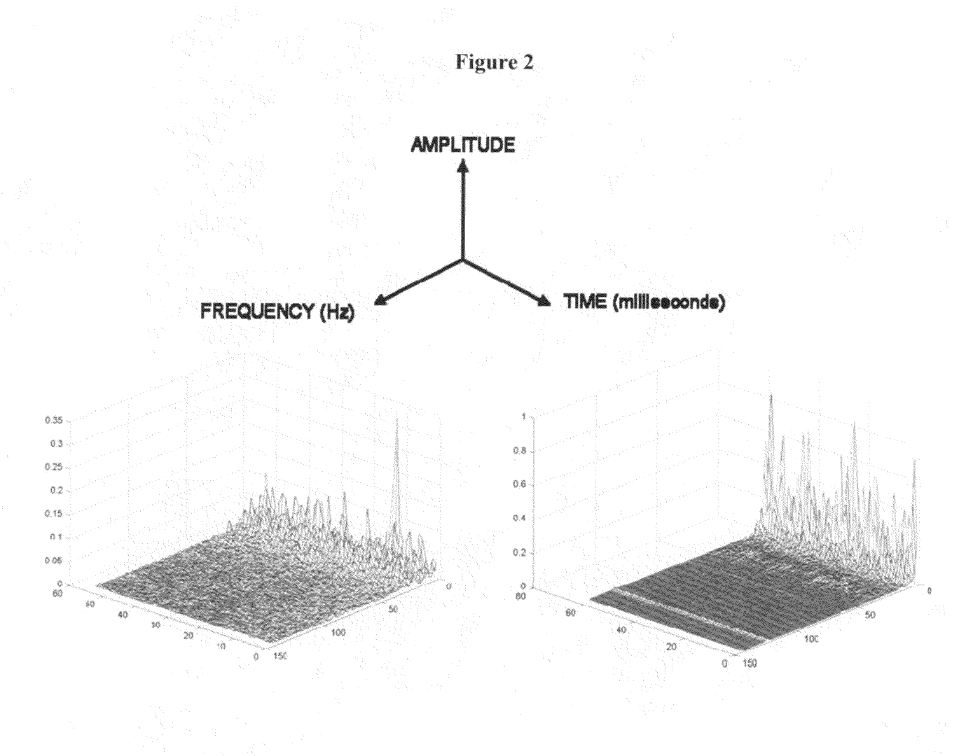

Cortical synchrony between temporal regions was observed in the patients with traumatic brain injuries at the lower frequencies (delta and theta) and as electroencephalogram frequency increased, cortical synchrony became evident in both patients and controls (FIG. 3). Synchrony (darkest red, R=1, maximal synchrony) emerges as frequency increases. The bar represents the graduation from maximal synchrony (dark red) to maximal desynchrony (dark blue, R=0, no synchrony). The patient exhibits area of temporal lobe synchrony at the lower frequencies (3 and 6 Hz), while the control subject does not. The patient had bilateral basal frontal and temporal lobe injuries on brain CT. In the delta frequency, 3 Hz±2, control subjects have a mean R value of 0.45 (range: 0.33 to 0.582) compared to TBI patients who have a mean R value of 0.76 (range: 0.584 to 0.86). Younger control subjects tend to have lower R values, while adolescents have R values in the upper range. The TBI and control group R values are significantly different, p=0.00012.

Increased synchronization at the lower beta frequency on the first electroencephalogram in those cortical areas associated with sites of focal injury was seen on the first computerized tomography scan: subdural hemorrhage, subarachnoid hemorrhage, intracerebral hematomae and contusions. Twelve of the 17 patients had focal cortical injuries (Table 2). The second electroencephalograms were also analyzed and compared to the patients' subsequent computerized tomography scans. Resolution of the primary injury was associated with decreased synchronization in the corresponding electroencephalogram channel, or loss of synchronization if the lesion completely resolved. If the lesion persisted, the electroencephalogram synchrony was still present in the corresponding EEG channels overlying the affected brain regions. There were no cases of patients with evolving lesions.

TABLE 2

| | EEG Channel | Computerized Tomography scan |
|---|---|---|
| Patient | Synchrony | findings |
| 1 | F8 | Right frontal hematoma |
| 2 | F4, FP2 | Right frontal hematoma |
| 3 | F4, F8, FP2 | Right frontal subdural hematoma |
| 4 | F4, F8, FP2, P4, T4, | Bilateral contusions in frontal, |
| | T6, O2/F3, FP1, P3, | temporal, parietal & occipital lobes |
| | T3, T5 | |
| 5 | F7, F3, FP2, T4, T5, | Basal frontal & temporal contusions |
| | T6 | |
| 6 | F4, F8 | Left and right frontal, Right temporal |
| 7 | T4 | Right frontal, right temporal subdural |
| | | hematoma |
| 8 | P3 | Left occipital-parietal hematoma |
| 9 | T4, T6, F4, P4 | Right fronto-temporal-parietal subdural |
| | | hematoma |
| 10 | P3, O1, F4 | Left occipital-parietal and right frontal |
| | | subdural hematoma |
| 11 | FP1, P3 | Left frontal- parietal subarachnoid |
| | | hemorrhage |
| 12 | F8, FP2 | Right frontal extra axial |

For each patient the EEG synchrony (S values) of the individual channels were compared with corresponding channels of age matched controls using the Student t test. Patient channels that were statistically different from those of control subjects (0.00001<p<0.001) were recorded and corresponding brain regions identified. These channels and brain regions were then compared to the patients' CT scan reports and were found to correlate to sites of primary injury. This was seen in the twelve patients with primary injuries of the cortex. Patients that had additional diffuse axonal injury (DAI) had other areas of cortical synchrony that were not apparent on CT scan.

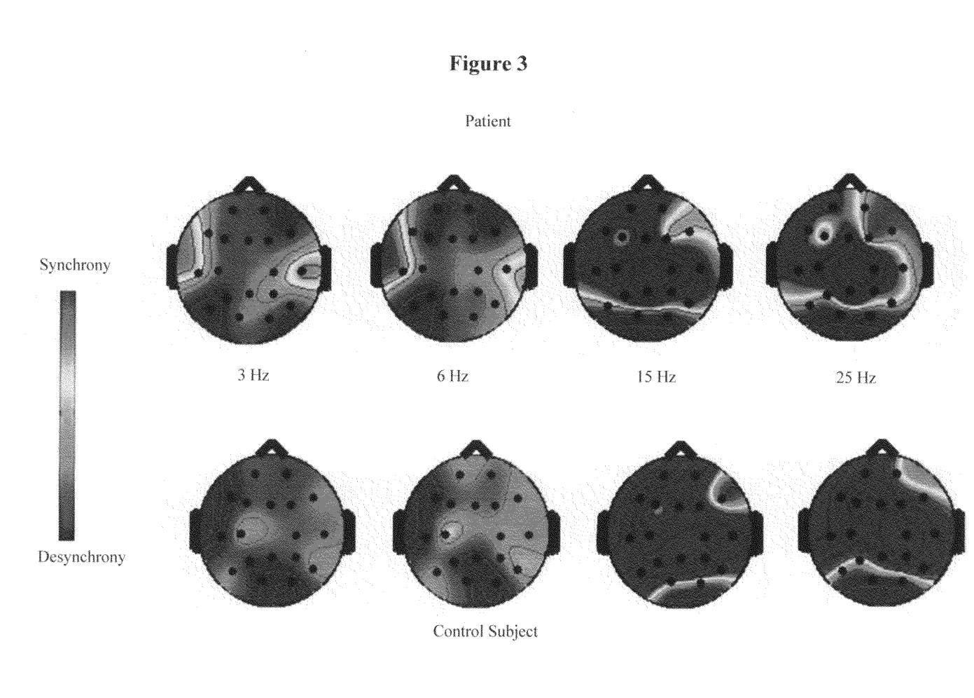

FIG. 4 demonstrates the placement of the EEG electrodes on a patient. Even numbers appear on the right side of the scalp while corresponding regions on the left side are designated with odd numbers and midline electrodes are represented by “Z”. The electrodes correspond to the following brain regions: FP=fronto-parietal; F=Frontal; C=Central; T=Temporal; P=Parietal and O=Occipital.

Interestingly, some common EEG synchrony patterns were found between adjacent electrodes (e g. between F7 and FP1 electrodes, adjacent areas in the left frontal lobe) that were present at all ages in both patients and controls.

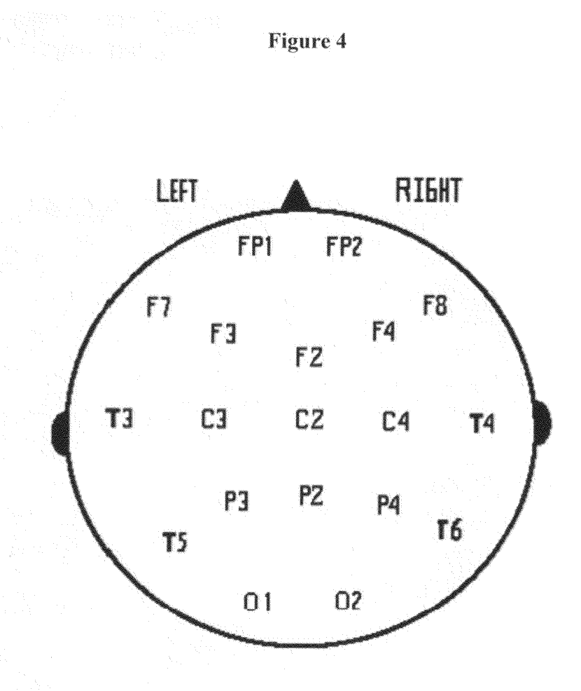

While electroencephalogram synchrony demonstrated correlation with static lesions, temporal variability was utilized to evaluate overall brain function over time. Visual inspection of the synchrony plots showed temporal fluctuations between synchrony and desynchrony, producing visually variable patterns in most patients and all controls (FIG. 5). In the synchrony graphs (bottom) mean synchrony [average of synchrony between each channel pair over a 10 millisecond epoch (y axis) at delta frequency (3 Hz±2 Hz)] of the raw EEG traces (top) are shown. These graphs show the variable pattern between synchrony (darkest red) and desynchrony (darkest blue) among EEG channels. Patient A, had a good outcome (PCPC=1). In comparison, Patient B with poor outcome (PCPC=4), has a pattern that demonstrates uniformity across all channels over time. The EEG of a control subject has a similar pattern to that of Patient A with continual fluctuation between synchrony to desynchrony.

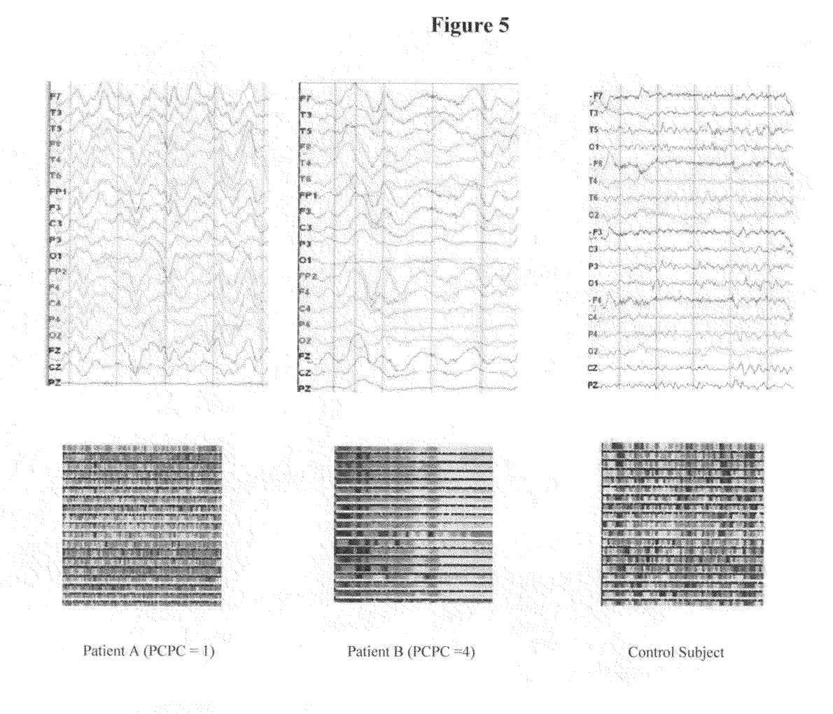

Patients with favourable functional outcome at 12 months post-injury and controls had similar ranges of temporal variability (FIG. 6). Patients with good outcome (PCPC 1 and 2; normal function to mild disability) and controls have similar distribution of EEG synchrony temporal variability. Patients with bad outcomes (PCPC 3 to 6, moderate disability to death) all have similar distributions and are found at the lower end of the scale which is in the order of 10⁻⁶.

When the temporal variability of the synchrony of the second electroencephalogram of each patient with traumatic brain injury was compared to the temporal variability of the synchrony of his/her first electroencephalogram, changes in 11 of the 17 patients were seen. Three of the patients had only one electroencephalogram taken as they were discharged from hospital before a second EEG could be obtained and therefore no comparison was possible. Temporal variability in the delta frequency range (3 Hz±2 Hz) increased significantly between the first and second electroencephalograms in those patients whose Glasgow coma scale (GCS) score increased (Table 3). Using linear regression, the correlations between i) Glasgow coma scale score, ii) age and iii) the temporal variability of the electroencephalogram at the delta frequency and the Pediatric Cerebral Performance Category (PCPC) score at 12 months post-injury were 0.576, 0.178 and 0.75 respectively. A persistent decrease in the variability in the synchronization pattern with no improvement in the Glasgow Coma Scale score was associated with unfavourable outcome post-injury.

The youngest control subjects (<2 years old) had the lowest temporal variability; particularly, the youngest control subject (11 months old) had significantly lower temporal variability compared to the other control subjects. This reflects age related brain development.

TABLE 3

| | Glasgow | Glasgow | | Change in |
|---|---|---|---|---|
| | Coma | Coma | | Temporal |
| Patient | Scale 1 | Scale 2 | p value | variability |
| 1 | 6 | 10 | 0.00002 | Increase |
| 2 | 15 | 15 | 0.264 | No change |
| 3 | 14 | 15 | 0.4056 | No change |
| 4 | 7 | 7 | 0.001 | Increase |
| 5 | 5 | 5 | 0.0099 | Decrease |
| 6 | 7 | 11 | 0.001 | Increase |
| 7 | 3 | 15 | 0.000001 | Increase |
| 8 | 13 | 15 | 0.001 | Increase |
| 9 | 15 | 15 | 0.2939 | No change |
| 10 | 10 | — | — | — |
| 11 | Not | 3 | 0.418 | Increase |
| | assessed | | | |
| 12 | 8 | 13 | 0.0003 | Increase |
| 13 | 15 | — | — | — |
| 14 | 15 | — | — | — |

### Discussion

Synchrony and temporal variability add another dimension to visual interpretation of electroencephalograms and have potential diagnostic and prognostic value in children with traumatic brain injury. Electroencephalogram synchrony correlated with the site of the primary injury and the temporal variability of synchrony in the delta frequency correlated with the functional outcome at 12 months and increased as the patients' Glasgow coma scale score improved.

The amplitudes in synchrony and its temporal variability of electroencephalograms in patients were compared to those of control subjects. Studying control subjects was valuable as they reflect normal brain function and development. It is possible that some synchrony patterns are established early in brain development and persist throughout maturation and are necessary for the establishment of neuronal circuitry. Absence or alteration of these patterns can reflect pathology.

The utility of synchrony and temporal variability calculations from electroencephalography can potentially provide continuous monitoring and more frequent analysis of a patient than currently utilized methods, such as CT scan. In addition, the present method can be used to provide information on brain function even if the patient's clinical status or Glasgow coma scale score remains unchanged.

Electroencephalography is an important non-invasive evaluation tool in paediatric traumatic brain injury. Novel use of electroencephalogram synchrony analysis and the temporal variability of synchronization provide insight into brain function post traumatic brain injury. Cortical activity is necessary for synchrony to exist and fluctuations between synchrony and desynchrony likely represent phase transitions in brain dynamics and reflect normal brain activity.

### Example 2

### Patient Population and Clinical Data

Retrospective study was conducted where patients were included if they were admitted to the Critical Care Unit at the Hospital For Sick Children, Toronto, Canada in coma from any of the following: traumatic brain injury, cardiac arrest, drowning, stroke, shock and organ failure, metabolic disorder or infectious disease. They had to have had a minimum of 2 EEGs at least 24 hours apart. Consent was waived by the Research Ethics Board. The control group used for the prospective observational pilot study was again used for this retrospective study.

Information gathered on each patient included age, gender, etiology of coma, and Glasgow coma scale which was recorded on admission, at the time of each EEG and upon discharge (unless the patient died). EEG recordings

### EEG Recordings

Each EEG was 30 minute in length, using a standard 10-20 montage with Pz prime (Pz′) as the reference electrode. Bandpass width of 1-70 Hz and a 60 Hz notch filter was used with a sampling frequency of 499 Hz. Each EEG had been evaluated by a certified electroencephalographer.

### Outcome Measures

Outcome was measured in two ways:

i) Change in the Glasgow Coma Score (GCS), where an increase would be associated with emergence from coma and decrease would indicate persistence of a comatose state; and

ii) Phase synchrony analysis and calculation of the temporal variability of synchronization. The method of phase synchrony analysis and temporal variability of EEG synchrony calculation is described in Example 1.

### Statistical Analysis

Repeated measures analysis of variance evaluated the within subject variance for the four 10-second epochs of each patient EEG. Positive (PPV) and negative (NPV) predictive values, sensitivity and specificity were calculated for the ability of an increase in temporal and spatial variability of EEG synchronization to predict emergence from coma using the following table (3):

TABLE 3

| | Comatose | Awake |
|---|---|---|
| Decrease in variability | A | B |
| Increase in variability | C | D |

PPV=A/(A+B)

NPV=D/(C+D)

Sensitivity=A/(A+C)

Specificity=D/(B+D)

### Results

Patient data are summarized below.

TABLE 4

| Patient | Age | Gender | Coma etiology |
|---|---|---|---|
| 1 | 13 y | M | Traumatic Brain Injury |
| 2 | 15 y | M | Traumatic Brain Injury |
| 3 | 5 y | M | Cardiac arrest |
| 4 | 15 y | F | Cardiac arrest |
| 5 | 12 y | M | Intracranial hemorrhage |
| 6 | 15 y | M | Cerebral infarction |
| 7 | 13 y | M | Arteriovenous malformation |
| 8 | 3 y | F | Septic Shock |
| 9 | 11 y | F | Vasculitis |
| 10 | 4 y | M | Cardiac arrest |
| 11 | 12 y | F | Cardiac arrest |
| 12 | 21 m | F | Cardiac arrest |
| 13 | 15 y | M | Traumatic Brain Injury |
| 14 | 15 y | F | Cardiac arrest |
| 15 | 5 y | M | Cardiac arrest |
| 16 | 16 y | M | Diabetic ketoacidosis |
| 17 | 5 y | F | Cardiac arrest |
| 18 | 16 y | M | Traumatic Brain Injury |
| 19 | 9 y | M | Traumatic Brain Injury |
| 20 | 15 y | M | Cardiac arrest |
| 21 | 15 y | M | Drowning |

The timing between first and second EEGs varied (range: 24 to 288 hours). The EEGs of all of the patients in coma, regardless of etiology, had a predominance of slower frequencies in the delta (1 to 3 Hz) and theta (4 to 6 Hz) range whereas the control subject EEGs had a predominance of higher frequencies.

When the temporal variability of the synchrony of the second EEG of each patient was compared to the temporal variability of the synchrony of his/her first EEG, changes in all 21 patients were noted as shown below in Table 5 which is a summary of the temporal variability of the temporal lobes of 25 patients in coma (mixed etiology) in the delta frequency (3 Hz±2 Hz).

TABLE 5

| Patient | EEG1 | EEG2 | change | Coma |
|---|---|---|---|---|
| 1 | 0.60744 | 0.5589 | Decrease | Yes |
| 2 | 0.56568 | 0.70931 | Increase | Yes |
| 3 | 0.53036 | 0.51359 | Decrease | No |
| 4 | 0.53567 | 0.63161 | Increase | No |
| 5 | 0.61457 | 0.376653 | Decrease | Yes |
| 6 | 0.5177 | 0.52952 | Increase | No |
| 7 | 0.63783 | 0.58974 | Decrease | Yes |
| 8 | 0.67904 | 0.57696 | Decrease | No |
| 9 | 0.63811 | 0.51889 | Decrease | Yes |
| 10 | 0.6523 | 0.62675 | Decrease | No |
| 11 | 0.50477 | 0.440605 | Decrease | Yes |
| 12 | 0.394025 | 0.482251 | Increase | Yes |
| 13 | 0.56577 | 0.50822 | Decrease | Yes |
| 14 | 0.49876 | 0.57581 | Increase | No |
| 15 | 0.49913 | 0.60452 | Increase | No |
| 16 | 0.63895 | 0.478468 | Decrease | Yes |
| 17 | 0.47732 | 0.51516 | Increase | No |
| 18 | 0.63222 | 0.57002 | Decrease | No |
| 19 | 0.60048 | 0.57492 | Decrease | No |
| 20 | 0.57758 | 0.5799 | Increase | Yes |
| 21 | 0.67542 | 0.60254 | Decrease | Yes |
| 22 | 0.63404 | 0.58066 | Decrease | Yes |
| 23 | 0.58375 | 0.51545 | Decrease | Yes |
| 24 | 0.5327 | 0.53056 | Decrease | Yes |
| 25 | 0.58478 | 0.57088 | Decrease | Yes |

Temporal variability in the delta frequency range (3 Hz±2 Hz) increased between the first and second EEGs in those patients whose Glasgow coma scale score increased.

We claim:

1. A method of identifying a site of brain injury in a pediatric patient comprising the steps of:
a) obtaining an electroencephalogram signal on the patient; and
b) evaluating phase synchronization of the signal obtained, wherein a determination of synchrony in the signal of at least 2 standard deviations greater than the mean synchrony of a control is indicative of a site of brain injury.

2. The method as defined in claim 1, wherein the electroencephalogram signal is obtained over a time period of about 15-45 minutes.

3. The method as defined in claim 2, wherein the time period is about 25-35 minutes.

4. The method as defined in claim 1, wherein the electroencephalogram signal is recorded at an acquisition rate of at least about 150 Hz.

5. The method as defined in claim 1, wherein phase synchronization is evaluated in a frequency range of about 1 to 30 Hz.

6. The method as defined in claim 5, wherein the frequency range is about 1 to 5 Hz.

7. A method of prognosis in a pediatric patient in a coma comprising the steps of:
a) obtaining a first electroencephalogram signal on the patient subsequent to the onset of coma;
b) obtaining a second electroencephalogram signal on the patient at a time subsequent to obtaining the first signal;
c) evaluating phase synchronization patterns in the first and second signals, or in a portion of the first and second signals; and.
d) calculating the temporal variability of the synchronized patterns, wherein increased variability in the signal over time is indicative of an improvement in the patient.

8. The method as defined in claim 7, wherein the first electroencephalogram signal is obtained at a time following onset of coma of up to about 5 days.

9. The method as defined in claim 7, wherein the second electroencephalogram signal is obtained at a time subsequent to the first electroencephalogram signal of up to about 7 days.

10. The method as defined in claim 7, wherein the first electroencephalogram signal is obtained within about 24-48 hours following onset of coma, and the second electroencephalogram signal is obtained within about 48-72 hours following onset of coma.

11. The method as defined in claim 7, wherein an increase in the temporal variability of the synchronized patterns in the range of about 10%-100% over time is indicative of an improvement in the patient.

12. The method as defined in claim 7, wherein the first and second electroencephalogram signals are each obtained over a time period of about 15-45 minutes.

13. The method as defined in claim 12, wherein the time period is about 25-35 minutes.

14. The method as defined in claim 7, wherein the first and second electroencephalogram signals are each recorded at an acquisition rate of at least about 150 Hz.

15. The method as defined in claim 7, wherein phase synchronization is evaluated at a frequency range of about 1 to 30 Hz.

16. The method as defined in claim 15, wherein the frequency range is about 1 to 5 Hz.

17. A method of monitoring the prognosis of a pediatric patient in a coma comprising the steps of:
a) obtaining an electroencephalogram signal on the patient at various times subsequent to the onset of coma;
b) evaluating phase synchronization patterns between electroencephalogram signals obtained at two different times;
d) calculating the temporal variability of the synchronized patterns, wherein increased variability in the electroencephalogram signal over time is indicative of an improvement in the patient.

18. The method as defined in claim 17, wherein the phase synchronization is evaluated at a frequency range of about 1 to 5 Hz.
